(19) United States
(12) Patent Application Publication
(10) Pub. No.: US 2022/0338746 A1
(43) Pub. Date: Oct. 27, 2022

(54) PULSE SENSOR AND PULSE SENSING SYSTEM

(71) Applicant: Au Optronics Corporation, Hsinchu (TW)

(72) Inventors: Ming-I Huang, Hsinchu (TW); Yu-Jung Liu, Hsinchu (TW); Chien Cheng Wang, Hsinchu (TW)

(73) Assignee: Au Optronics Corporation, Hsinchu (TW)

(21) Appl. No.: 17/519,570

(22) Filed: Nov. 4, 2021

(30) Foreign Application Priority Data
Apr. 21, 2021 (TW) 110114282

(51) Int. Cl. A61B 5/0245

(52) U.S. Cl. CPC A61B 5/0245; A61B 2562/0247; A61B 2562/046

(57) ABSTRACT

A pulse sensor and a pulse sensing system are provided. The pulse sensor includes a pressure sensing circuit, a reference circuit, and an output circuit. The pressure sensing circuit may sense a pulse vibration to generate a sensing signal. The reference circuit may generate a reference signal according to a base signal. A first input terminal of the output circuit is coupled to the pressure sensing circuit. A second input terminal of the output circuit is coupled to the reference circuit. The output circuit generates a pulse sensing current at an output terminal of the output circuit according to a difference between the sensing signal and the reference signal.

### CROSS-REFERENCE TO RELATED APPLICATION

This application claims the priority benefit of Taiwan application serial no. 110114282, filed on Apr. 21, 2021. The entirety of the above-mentioned patent application is hereby incorporated by reference herein and made a part of this specification.

### BACKGROUND

### Technical Field

The disclosure relates to a sensor and a sensing system, and more particularly to a pulse sensor and a pulse sensing system.

### Description of Related Art

When an existing pulse sensor senses human pulses, if through a piezoelectric material, sensing signals are often affected by noise introduced through human body coupling, resulting in deviation of sensing results. Therefore, how to eliminate the noise has become an important issue during pulse sensing.

### SUMMARY

The disclosure provides a pulse sensor and a pulse sensing system, which may eliminate noise sensed by the pulse sensor.

The pulse sensor of the disclosure includes a pressure sensing circuit, a reference circuit, and an output circuit. The pressure sensing circuit may sense a pulse vibration to generate a sensing signal. The reference circuit may generate a reference signal according to a base signal. A first input terminal of the output circuit is coupled to the pressure sensing circuit. A second input terminal of the output circuit is coupled to the reference circuit. The output circuit generates a pulse sensing current at an output terminal of the output circuit according to a difference between the sensing signal and the reference signal.

The pulse sensing system of the disclosure includes a sensing array formed by multiple pulse sensors. The pulse sensor includes a pressure sensing circuit, a reference circuit, and an output circuit. The pressure sensing circuit may sense a pulse vibration to generate a sensing signal. The reference circuit may generate a reference signal according to a base signal. A first input terminal of the output circuit is coupled to the pressure sensing circuit. A second input terminal of the output circuit is coupled to the reference circuit. The output circuit generates a pulse sensing current at an output terminal of the output circuit according to a difference between the sensing signal and the reference signal.

Based on the above, the pulse sensor and the pulse sensing system of the disclosure may receive in-phase noise through the pressure sensing circuit and the reference circuit, and may generate the pulse sensing current after deducting the noise to eliminate the influence of the noise on pulse sensing.

### BRIEF DESCRIPTION OF THE DRAWINGS

FIG. 1 is a block diagram of a pulse sensor according to the embodiments of the disclosure.

FIG. 2A is a schematic diagram of a pulse sensor according to the embodiments of the disclosure.

FIG. 2B is a schematic diagram of operation waveforms of a pulse sensor according to the embodiments of the disclosure.

FIG. 3 is a schematic diagram of a pulse sensing system according to the embodiments of the disclosure.

FIG. 4A is a schematic layout diagram of a pulse sensing system according to the embodiments of the disclosure.

FIG. 4B is a cross-sectional diagram taken along a line in FIG. 4A.

FIG. 4C is a cross-sectional diagram taken along a line in FIG. 4A.

### DESCRIPTION OF THE EMBODIMENTS

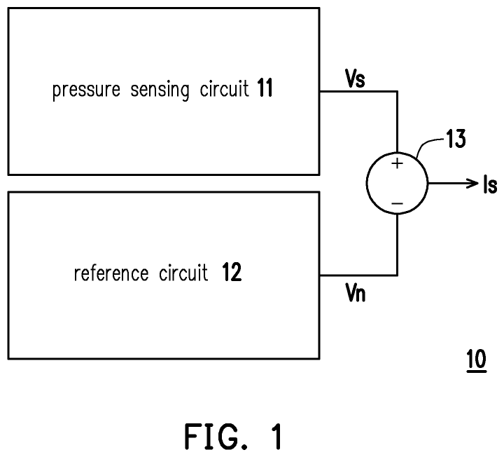

FIG. 1 is a block diagram of a pulse sensor 10 according to the embodiments of the disclosure. The pulse sensor 10 may be used to be attached to the skin of a user and sense a pulse according to a skin vibration. The pulse sensor 10 includes a pressure sensing circuit 11, a reference circuit 12, and an output circuit 13. The pressure sensing circuit 11 may sense a pulse vibration to generate a sensing signal Vs. The reference circuit 12 may generate a reference signal Vn based on a base signal. The output circuit 13 has two input terminals, respectively coupled to the pressure sensing circuit 11 and the reference circuit 12. The output circuit 13 may generate a pulse sensing current Is according to a difference between the sensing signal Vs and the reference signal Vn.

In detail, the pressure sensing circuit 11 may be attached to the skin of the user to sense the skin vibration for generating the sensing signal Vs. However, the sensing signal Vs generated by the pressure sensing circuit 11 includes not only components of pressure signals for sensing pressure but also components of noise signals formed by noise through human body coupling. In this way, the noise often affects pulse sensing results. Therefore, the reference circuit 12 is disposed in the pulse sensor 10. The reference circuit 12 is disposed near the pressure sensing circuit 11. The reference circuit 12 may sense the noise coupled through the human body and receive an electrical signal (i.e., the base signal) on the skin of the user, generating the reference signal Vn through the change of the base signal, such that the reference signal Vn obtained by the reference circuit 12 may be in-phase with and have the same amplitude as the noise in the sensing signal Vs obtained by the pressure sensing circuit 11. In this way, the pulse sensor 10 may remove the noise in the sensing signal Vs by deducting the reference signal Vn from the sensing signal Vs, and may allow the generated pulse sensing current Is to be accurate and not be interfered by the noise, thereby effectively improving the sensing quality of the pulse sensor 10.

In an embodiment, the noise introduced and coupled through the user may be, for example, a frequency of a household electrical system (60 Hz). In this case, since the frequency of the household power system is close to the pulse frequency of the user or overlaps with the pulse frequency of the user, it is usually difficult to separate the noise signal and the pulse signal by filtering. Therefore, with the pressure sensing circuit 11 and the reference circuit 12 disposed in the pulse sensor 10 of the disclosure, both of which may receive the in-phase noise, the noise interference may ideally be completely removed through the pulse sensing current Is generated by deducting the reference signal Vn from the sensing signal Vs sensed by the pressure sensing circuit 11, thereby effectively improving the accuracy of pulse sensing.

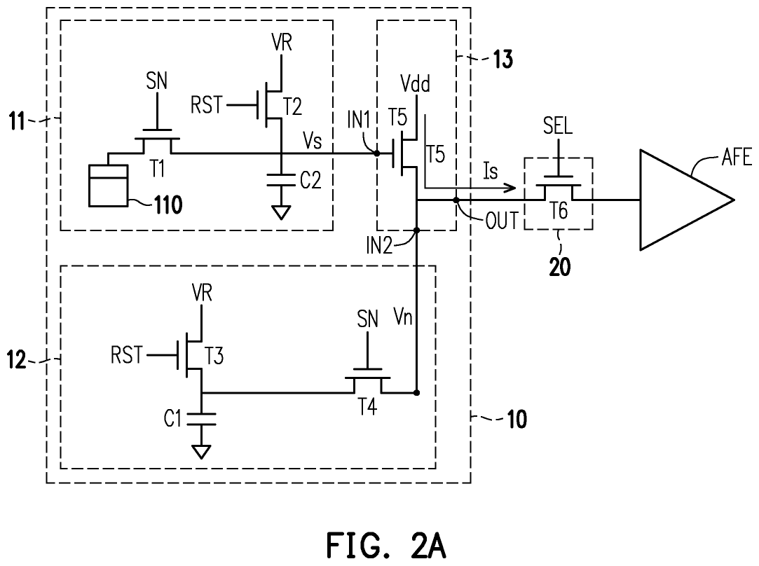

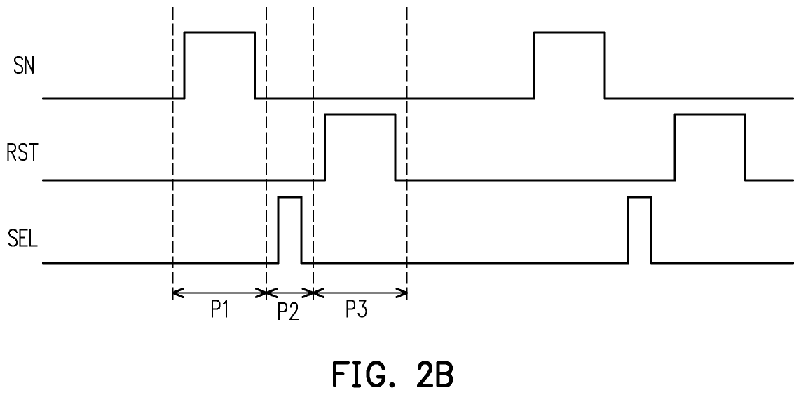

FIG. 2A is a schematic circuit diagram of the pulse sensor 10 according to the embodiments of the disclosure. The pulse sensor 10 is coupled to a drive switch 20 and provides the pulse sensing current Is to an interface circuit AD through the drive switch 20. In general, the pulse sensor 10 may sense the pulse pressure on the skin of the user to generate the sensing signal Vs and sense the reference signal Vn generated by the noise coupled through the user, and may output the pulse sensing current Is based on the difference between the sensing signal Vs and the reference signal Vn. The drive switch 20 may determine whether to output the pulse sensing current Is according to an output selection signal SEL. The interface circuit AD may convert the pulse sensing current Is into voltage for output and analysis.

In detail, the pressure sensing circuit 11 is coupled to an input terminal IN1 of the output circuit 13. The pressure sensing circuit 11 includes a piezoelectric conversion element 110, switches T1, T2, and a capacitor C2. The piezoelectric conversion element 110 may be attached to the skin of the user to generate the sensing signal Vs. A first terminal of the switch T1 is coupled to the piezoelectric conversion element 110, a second terminal of the switch T1 is coupled to the input terminal IN1 of the output circuit 13, and a control terminal of the switch T1 receives a scan signal SN. A first terminal of the switch T2 receives a reference voltage VR, a second terminal of the switch T2 is coupled to the second terminal of the switch T1, and a control terminal of the switch T2 receives a reset signal RST. The capacitor C2 is coupled between the second terminal of the switch T1 and a reference voltage Gnd. The capacitor C2 may be used to store and output the sensing signal Vs.

The reference circuit 12 is coupled to an input terminal IN2 of the input circuit 13. The reference circuit 12 includes a capacitor C1 and switches T3, T4. The capacitor C1 is for generating the reference signal Vn according to the noise introduced and coupled through the user. A first terminal of the switch T3 receives the reference voltage VR, a second terminal of the switch T3 is coupled to the capacitor C1, and a control terminal of the switch T3 receives the reset signal RST. A first terminal of the switch T4 is coupled to the second terminal of the switch T3, a second terminal of the switch T4 is coupled to the input terminal IN2 of the output circuit 13, and a control terminal of the switch T4 receives the scan signal SN. In detail, one terminal of the capacitor C1 is coupled to the second terminal of the switch T3, and the other terminal of the capacitor C1 may be coupled to the ground through the user when the pulse sensor 10 is attached to the skin of the user. In this way, when the noise is coupled and introduced to the pulse sensor 10 through the human body, the voltage change caused by the noise is sensed by the reference circuit 12 through the capacitor C1.

The output circuit 13 receives the sensing signal Vs and the reference signal Vn respectively through the input terminals IN1 and IN2, and generates the pulse sensing current Is at an output terminal OUT. The output circuit includes a switch T5. A first terminal of the switch T5 receives a reference voltage Vdd, a control terminal of the switch T5 is coupled to the input terminal IN1 of the output circuit 13 to receive the sensing signal Vs, and a second terminal of the switch T5 is coupled to the input terminal IN2 of the output circuit 13 to receive the reference signal Vn. According to the difference between the sensing signal Vs and the reference signal Vn, the switch T5 provides the pulse sensing current Is to the output terminal OUT of the output circuit 13 through the second terminal of the switch T5.

The drive switch 20 is coupled to the pulse sensor 10. The drive switch 20 may determine whether to output the pulse sensing current Is according to the output selection signal SEL. The drive switch 20 includes a switch T6. A first terminal of the switch T6 is coupled to the output terminal OUT of the output circuit 13 for receiving the pulse sensing current Is, a second terminal of the switch T6 is coupled to the interface circuit AD, and a control terminal of the switch T6 receives the output selection signal SEL.

In an embodiment, the switches T1 to T6 illustrated in FIG. 2A may be, for example, N-type metal oxide silicon field-effect transistors (MOSFET), which may be enabled to turn on the connection between the first terminal (such as a drain) and the second terminal (such as a source) when a voltage signal of a high voltage level is received at the control end (such as a gate), and may be cut off to disconnect the connection between the first terminal and the second terminal when a voltage signal of a low voltage level is received at the control terminal, but this disclosure is not limited to the above. For example, P-type MOSFETs, bipolar junction transistors (BJT), or other suitable elements also fall within the scope of this disclosure.

FIG. 2B is a schematic diagram of operation waveforms of a pulse sensor according to the embodiments of the disclosure. FIG. 2B shows the operation waveforms of the scan signal SN, the reset signal RST, and the output selection signal SEL in time intervals P1, P2, and P3. The time intervals P1 to P3 may be executed cyclically and repeatedly for the pulse sensor to repeat the process of sensing pulses of a user. The operation waveforms shown in FIG. 2B may be applied to the pulse sensor 10 shown in FIG. 2A. Therefore, please refer to FIG. 2A and FIG. 2B together to understand the operation description of the pulse sensor 10 in the following paragraphs.

First, in the time interval P1, the scan signal SN is switched to the high voltage level. In this way, the switch T1 in the pressure sensing circuit 11 is enabled and turned on, providing the sensing signal Vs to the input terminal IN1 of the output circuit 13. In addition, the switch T4 in the reference circuit 12 is also enabled and turned on, providing the reference signal Vn to the input terminal IN2 of the output circuit 13.

In detail, the switch T5 in the output circuit 13 may be an N-type MOSFET. By receiving the sensing signal Vs at the control terminal (such as a gate) and receiving the reference signal at the second terminal (such as a source), the switch T5 may generate the pulse sensing current Is according to a voltage difference value between the control terminal and the second terminal thereof. In this way, components of the noise in the sensing signal Vs may be eliminated when the reference signal Vn is deducted from the sensing signal Vs, and thus the pulse sensing current Is not affected by the noise is generated at the output terminal OUT to offset the noise interference.

Next, in the time interval P2, the scan signal SN is switched back to the low voltage level, and the output selection signal SEL is switched to the high voltage level. In this way, the drive switch 20 may be enable and turned on by the output selection signal SEL, thereby providing the pulse sensing current Is to the interface circuit AD. The interface circuit AD may be, for example, an analog to digital converter (ADC). The interface circuit AD may convert the pulse sensing current Is into a voltage result in a digital form for output, so as to facilitate the subsequent pulse analysis.

In the time interval P3, the output selection signal SEL is switched back to the low voltage level, and the reset signal is switched to the high voltage level. In this way, the switch T2 in the pressure sensing circuit 11 and the switch T3 in the reference circuit 12 may be enabled and turned on. The reference voltage VR may be provided to the capacitors C1 and C2, thereby resetting electric charges on the capacitors C1 and C2.

Therefore, the pulse sensor 10 may generate the sensing signal Vs for pulse vibrations on the skin of the user and sense the reference signal Vn generated by the noise coupled through the user at the same time, and may generate the pulse sensing current Is according to the difference between the sensing signal Vs and the reference signal Vn. In this way, the pulse sensor 10 may remove the noise in the sensing signal Vs by deducting the reference signal Vn from the sensing signal Vs, and may allow the generated pulse sensing current Is to be accurate and not to be interfered by the noise, thereby effectively improving the sensing quality of the pulse sensor 10.

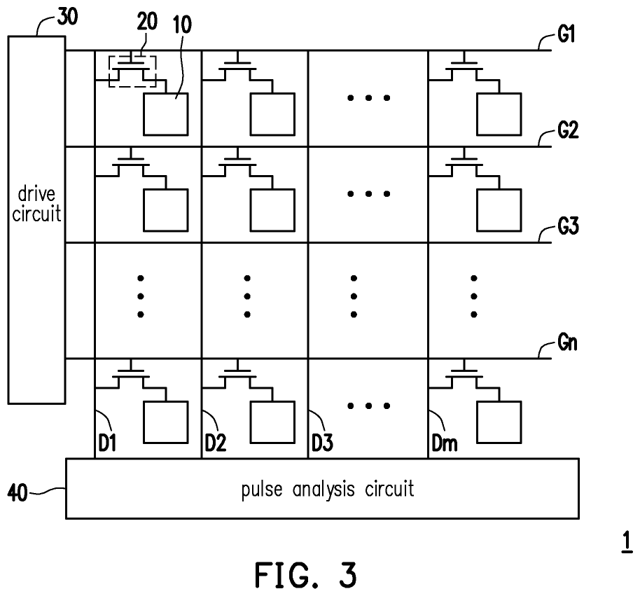

FIG. 3 is a schematic diagram of a pulse sensing system 1 according to the embodiments of the disclosure. The pulse sensing system 1 includes the pulse sensor 10, the drive switch 20, a drive circuit 30, and a pulse analysis circuit 40. The pulse sensor 10 shown in FIG. 3 may be, for example, the pulse sensor 10 in FIG. 1 or FIG. 2A. Therefore, for relevant description, reference may be made to the relevant paragraphs above, and details are not described herein.

All the pulse sensors 10 and the drive switches 20 may form a sensing array together. Each of the pulse sensors 10 may be connected to the corresponding drive switch 20 and may provide a pulse sensing current generated by sensing pulses of a user to the drive switch 20. The drive switch 20 is coupled to one of scan lines G1 to Gn and one of data lines D1 to Dm. The drive switch 20 may selectively provide the pulse sensing current to the corresponding data line according to the voltage of the corresponding scan line. In an embodiment, the sensing array formed by the pulse sensors 10 may be disposed on a sensing patch. The sensing patch may be attached to the skin of the user for sensing pulses of the user in a certain region on the skin, and may convert pulse signals in the time domain sensed in the region into pulse signals in the frequency domain through an operational circuit for operations to generate pulse sensing information.

Overall, the drive circuit 30 may generate output selection signals which are sequentially enabled and provide the output selection signals to the corresponding scan lines G1 to Gn to sequentially enable each row of the sensing array. In this way, the drive switch 20 on each row may be enabled and turned on, thereby providing the pulse sensing current generated by the pulse sensor 10 to the data lines D1 to Dm for the pulse analysis circuit 40 to analyze the pulses of the user.

In detail, the pulse analysis circuit 40 may receive the pulse sensing current and convert the pulse sensing current into voltage information. Furthermore, the pulse analysis circuit 40 may include, for example, the interface circuit AD shown in FIG. 2A and/or other logical operation digit circuits. The pulse analysis circuit 40 may analyze the voltage information of the pulses of user. For example, the pulse analysis circuit 40 may convert the received pulse sensing current from the time domain into the frequency domain for operations and generate the pulse sensing information of a pulse spectrogram of the user.

In an embodiment, the noise transmitted and coupled through the user may be, for example, a frequency of a household electrical system (60 Hz). In this case, since the frequency of the household power system is close to the pulse frequency of the user, it is usually difficult to separate the noise signal and the pulse signal by filtering. Therefore, with the pressure sensing circuit 11 and the reference circuit 12 disposed in the pulse sensor 10 of the disclosure, both of which may receive the in-phase noise, the noise interference may ideally be completely removed through the pulse sensing current Is generated by the voltage difference between the pressure sensing circuit 11 and the reference circuit 12, thereby effectively improving the accuracy of pulse sensing.

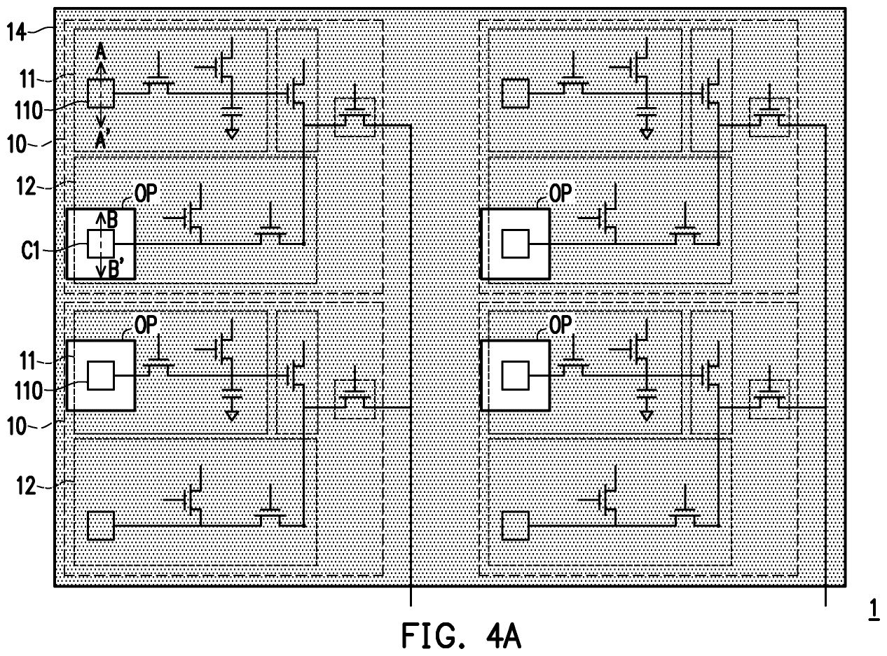

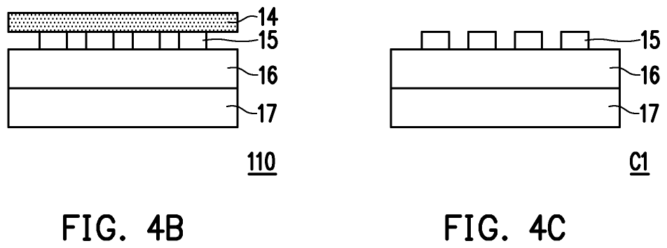

FIG. 4A is a schematic layout diagram of the pulse sensing system 1 according to the embodiments of the disclosure. For the convenience of description, reference signs of some components in the pulse sensing system 1 shown in FIG. 4A are omitted.

As shown in FIG. 4A, the pulse sensing system 1 includes a piezoelectric material layer 14, and the piezoelectric material layer 14 has multiple openings OP. The opening OP of the piezoelectric material layer 14 may expose the capacitor C1 of the reference circuit 12, and the piezoelectric material layer 14 may cover the pressure circuit 11 to form a piezoelectric conversion element 110.

FIG. 4B is a cross-sectional diagram taken along a line AA′ in FIG. 4A. FIG. 4B shows the piezoelectric material layer 14, an electrode layer 15, an array layer 16, and a substrate 17. The piezoelectric material layer 14 may be formed of, for example, organic, inorganic, or composite materials. The inorganic piezoelectric materials include piezoelectric crystals and piezoelectric ceramics, such as barium titanate (BaTiO3), lead zirconate titanate (PZT), lithium niobate (LiNbO3), or other suitable materials. The organic materials include polymers, such as polyvinylidene fluoride (PVDF) or other suitable materials. The composite materials may be, for example, a combination of ceramic lead zirconate titanate and polymer polyvinylidene fluoride or a combination of other suitable materials. The electrode layer 15 may include, for example, an electrode structure formed of indium tin oxide (ITO) or other suitable transparent conductors. The array layer 16 includes, for example, switches or transistor elements in the pressure sensing circuit 11 and the reference circuit 12. The substrate 17 may be, for example, a glass substrate, a polyimide (PI) substrate, or a substrate formed of other suitable materials.

In detail, the piezoelectric conversion element 110 may include the piezoelectric material layer 14 and the electrode structure in the electrode layer 15. The piezoelectric conversion element 110 may be formed by the piezoelectric material layer 14 covering on the electrode layer 15 and may be disposed on the array layer 16 and the substrate 17. When the pulse sensor 10 is attached to the skin of the user, the piezoelectric conversion element 110 may convert pulse pressure into electrical signals through the piezoelectric material layer 14 and provide the sensing signal Vs to circuits or switch elements disposed on the array layer 16 for signal processing and operations through the electrode structure in the electrode layer 15.

FIG. 4C is a cross-sectional diagram of the capacitor C1 according to the embodiments of the disclosure. FIG. 4C shows the electrode layer 15, the array layer 16, and the substrate 17. In detail, the capacitor C1 may include the electrode structure in the electrode layer 15. The capacitor C1 may be formed by disposing the opening OP of the piezoelectric material layer 14 on the electrode layer 15. More specifically, the opening OP of the piezoelectric material layer 14 is disposed on the electrode structure of the electrode layer 15 and is disposed on the array layer 16 and the substrate 17, such that the opening OP of the piezoelectric material layer 14 exposes the electrode structure in the electrode layer 15. In this way, when the pulse sensor 10 is attached to the skin of the user, one terminal of the capacitor C1 may receive electric signals (i.e., the base signal) from the user, and the other terminal of the capacitor C1 reflects the change of the electric signals. Therefore, the capacitor C1 may generate the reference signal Vn based on the base signal received on the skin of the user, and may sense the noise coupled through the user since the base signal and the reference signal Vn have the same voltage change. Next, the reference signal Vn may be provided to the circuits or the switch elements disposed on the array layer 16 for signal processing and operations.

In an embodiment, since the piezoelectric conversion element 110 of the pressure sensing circuit 11 may be disposed near the capacitor C1 of the reference circuit 12, the piezoelectric conversion element 110 and the capacitor C1 may receive the in-phase noise, and then the output circuit 12 performs subtraction to remove the noise and output the pulse sensing current.

Of course, a person having ordinary skill in the art may modify the above content according to different system and design requirements to achieve the same or similar objectives. For example, the layout of the pulse sensing system 1 is not limited to the layout structure shown in FIG. 4A. In the pulse sensing system 1, one opening OP of the piezoelectric material layer 14 may expose more than one capacitor C1, which means one opening OP may expose two, four, or more capacitors C1. Alternatively, on the contrary, the piezoelectric material layer 14 may be disposed within a small area to cover a region to be disposed as the piezoelectric conversion element 110, with most of the other region reserved as the opening OP, which is also a possible implementation. In this way, such flexible implementation may effectively reduce layout design complexity of the pulse sensing system 1.

In summary, with the pressure sensing circuit and the reference circuit disposed in the pulse sensor of the disclosure, both of which may receive the in-phase noise, the noise interference may ideally be completely removed through the pulse sensing current generated by deducting the reference signal from the sensing signal sensed by the pressure sensing circuit, and only the pulse sensing information is reserved. Therefore, pulse sensing accuracy may be effectively improved.

What is claimed is:

1. A pulse sensor, comprising:
a pressure sensing circuit, sensing a pulse vibration to generate a sensing signal;
a reference circuit, generating a reference signal according to a base signal; and
an output circuit, having a first input terminal, a second input terminal, and an output terminal, wherein the first input terminal of the output circuit is coupled to the pressure sensing circuit, the second input terminal of the output circuit is coupled to the reference circuit, and the output circuit generates a pulse sensing current at the output terminal of the output circuit according to a difference between the sensing signal and the reference signal.

2. The pulse sensor according to claim 1, wherein the pressure sensing circuit comprises a piezoelectric conversion element having a first electrode structure and a piezoelectric material layer disposed on the first electrode structure, and the piezoelectric conversion element is for being attached to a skin surface of a user to sense the pulse vibration to generate the sensing signal, wherein
the reference circuit comprises a first capacitor, the first capacitor has a second electrode structure, and the first capacitor is for being attached to the skin surface of the user to generate the reference signal according to the base signal.

3. The pulse sensor according to claim 2, wherein the pressure sensing circuit further comprises:
a first switch, having a first terminal, a second terminal, and a control terminal, wherein the first terminal of the first switch is coupled to the piezoelectric conversion element, the second terminal of the first switch is coupled to the first input terminal of the output circuit, and the control terminal of the first switch receives a scan signal;
a second switch, having a first terminal, a second terminal, and a control terminal, wherein the first terminal of the second switch receives a first reference voltage, the second terminal of the second switch is coupled to the second terminal of the first switch, and the control terminal of the second switch receives a reset signal; and
a second capacitor, coupled to the second terminal of the first switch for storing and outputting the sensing signal.

4. The pulse sensor according to claim 2, wherein the reference circuit further comprises:
a third switch, having a first terminal, a second terminal, and a control terminal, wherein the first terminal of the third switch receives a first reference voltage, the second terminal of the third switch is coupled to the first capacitor, and the control terminal of the third switch receives a reset signal; and
a fourth switch, having a first terminal, a second terminal, and a control terminal, wherein the first terminal of the fourth switch is coupled to the second terminal of the third switch, the second terminal of the fourth switch is coupled to the second input terminal of the output circuit, and the control terminal of the fourth switch receives a scan signal.

5. The pulse sensor according to claim 1, wherein the output circuit comprises a fifth switch having a first terminal, a second terminal, and a control terminal, the first terminal of the fifth switch receives a second reference voltage, the control terminal of the fifth switch receives the sensing signal, the second terminal of the fifth switch receives the reference signal, and the fifth switch provides the pulse sensing current to the output terminal of the output circuit through the second terminal of the fifth switch according to the difference between the sensing signal and the reference signal.

6. A pulse sensing system, comprising:
a sensing array, comprising a plurality of pulse sensors, each of the plurality of pulse sensors comprising:
a pressure sensing circuit, sensing a pulse vibration to generate a sensing signal;
a reference circuit, generating a reference signal according to a base signal; and
an output circuit, having a first input terminal, a second input terminal, and an output terminal, wherein the first input terminal of the output circuit is coupled to the pressure sensing circuit, the second input terminal of the output circuit is coupled to the reference circuit, and the output circuit generates a pulse sensing current at the output terminal of the output circuit according to a difference between the sensing signal and the reference signal.

7. The pulse sensing system according to claim 6, wherein the sensing array comprises:
a plurality of drive switches, respectively coupled to output terminals of the output circuits, wherein each of the plurality of drive switches is controlled by an output selection signal to respectively determine whether to output the pulse sensing current.

8. The pulse sensing system according to claim 7, further comprising:
a drive circuit, providing the output selection signals enabled in sequence to the sensing array; and
a pulse analysis circuit, wherein the plurality of pulse sensors receive the pulse sensing currents and analyze a frequency spectrum of the pulse sensing currents to generate pulse sensing information.

9. The pulse sensing system according to claim 6, comprising:
a plurality of electrode structures;
a piezoelectric material layer, disposed on the plurality of electrode structures, the piezoelectric material layer having a plurality of openings, wherein
the piezoelectric material layer covers a plurality of first electrode structures of the plurality of electrode structures to form a plurality of piezoelectric conversion elements in the pressure sensing circuits; and
the plurality of openings of the piezoelectric material layer expose a plurality of second electrode structures of the plurality of electrode structures to form a plurality of first capacitors in the reference circuits.

10. The pulse sensing system according to claim 9, wherein each of the pressure sensing circuits further comprises:
a first switch, having a first terminal, a second terminal, and a control terminal, wherein the first terminal of the first switch is coupled to the piezoelectric conversion element, the second terminal of the first switch is coupled to the first input terminal of the output circuit, and the control terminal of the first switch receives a scan signal;
a second switch, having a first terminal, a second terminal, and a control terminal, wherein the first terminal of the second switch receives a first reference voltage, the second terminal of the second switch is coupled to the second terminal of the first switch, and the control terminal of the second switch receives a reset signal; and
a second capacitor, coupled to the second terminal of the first switch for storing and outputting the sensing signal.

11. The pulse sensing system according to claim 9, wherein each of the reference circuits further comprises:
a third switch, having a first terminal, a second terminal, and a control terminal, wherein the first terminal of the third switch receives a first reference voltage, the second terminal of the third switch is coupled to the first capacitor, and the control terminal of the third switch receives a reset signal; and
a fourth switch, having a first terminal, a second terminal, and a control terminal, wherein the first terminal of the fourth switch is coupled to the second terminal of the third switch, the second terminal of the fourth switch is coupled to the second input terminal of the output circuit, and the control terminal of the fourth switch receives a scan signal.

12. The pulse sensing system according to claim 6, wherein each of the output circuits comprises a fifth switch having a first terminal, a second terminal, and a control terminal, the first terminal of the fifth switch receives a second reference voltage, the control terminal of the fifth switch receives the sensing signal, the second terminal of the fifth switch receives the reference signal, and the fifth switch provides the pulse sensing current to the output terminal of the output circuit through the second terminal of the fifth switch according to the difference between the sensing signal and the reference signal.
